(19) United States
(12) Patent Application Publication
(10) Pub. No.: US 2003/0105007 A1
(43) Pub. Date: Jun. 5, 2003

(54) PDGF-betabeta and fibronectin combined in a solid wound dressing for the treatment of wounds

(76) Inventors: Andre Beaulieu, Cap-Rouge (CA); Robert Paquin, St-Jean-Chrsostome (CA); Benoit Lariviere, St-Nicholas (CA)

Correspondence Address: OSTRAGER CHONG & FLAHERTY LLP, 825 THIRD AVE, 30TH FLOOR, NEW YORK, NY, 10022-7519, US

(21) Appl. No.: 10/145,678

(22) Filed: May 15, 2002

Related U.S. Application Data
(63) Continuation-in-part of application No. 10/049,992.
(63) A 371 of international application No. PCT/CA00/00953, filed on Feb. 19, 2002.
(63) Continuation-in-part of application No. 10/108,206, filed on Mar. 26, 2002.
(62) Division of application No. 09/862,971, filed on May 22, 2001, now abandoned.
(62) Division of application No. 09/245,785, filed on Feb. 5, 1999, now Pat. No. 6,251,859.
(62) Division of application No. 08/879,159, filed on Jun. 19, 1997, now Pat. No. 5,877,149.
(63) Continuation-in-part of application No. 08/488,253, filed on Jun. 7, 1995, now Pat. No. 5,641,483.
(63) Continuation-in-part of application No. 09/331,344, filed on Aug. 21, 1999, now Pat. No. 6,528,483.
(63) A 371 of international application No. PCT/CA97/00966, filed on Dec. 12, 1997.
(60) Provisional application No. 60/291,061, filed on May 15, 2001.
(60) Provisional application No. 60/182,412, filed on Feb. 14, 2000.
(60) Provisional application No. 60/149,958, filed on Aug. 20, 1999.

(51) Int. Cl. A61K 38/18; A61K 38/36

(52) U.S. Cl. 514/12

(57) ABSTRACT

This invention relates to the production of topical dosage forms containing human plasma fibronectin in combination with growth factors having human mitogenic or angiogenic activity, such as platelet-derived growth factor (PDGF-ββ), to promote wound healing in humans.

### RELATED APPLICATIONS

This application claims the priority of U.S. provisional Application Serial No. 60/291,061 filed May 15, 2001.

This application is a continuation-in-part of U.S. application Ser. No. 10/049,992 filed Feb. 19, 2002, which is the United States national phase application of International Application No. PCT/CA00/00953, as amended Feb. 19, 2002, which claims the priority of U.S. provisional Application Serial No. 60/182,412, filed Feb. 14, 2000 and U.S. provisional Application Serial No. 60/149,958, filed Aug. 20, 1999.

This application is also a continuation-in-part of U.S. application Ser. No. 10/108,206, filed Mar. 26, 2002, which is a divisional application of U.S. application Ser. No. 09/862,971, filed May 22, 2001, now abandoned, which is a divisional application of U.S. application Ser. No. 09/245,785, filed Feb. 5, 1999, which issued as U.S. Pat. No. 6,251,859. U.S. application Ser. No. 09/245,785 is a divisional application of U.S. application Ser. No. 08/879,159, filed Jun. 19, 1997, which issued as U.S. Pat. No. 5,877,149. U.S. application Ser. No. 08/879,159 is a continuation-in-part of U.S. application Ser. No. 08,488,253, filed Jun. 7, 1995, which issued as U.S. Pat. No. 5,641,483.

This application is a continuation-in-part application of U.S. application Ser. No. 09/331,344, filed Aug. 21, 1999, which is the United States national phase application of International Application No. PCT/CA97/00966, filed Dec. 12, 1998, which claims the priority of U.S. application Ser. No. 08/767,868, filed Dec. 17, 1996, which issued as U.S. Pat. No. 5,821,220. U.S. application Ser. No. 089/767,868 is a continuation-in-part of U.S. application Ser. No. 08/488,253, filed Jun. 7, 1995, which issued as U.S. Pat. No. 5, 641,483.

### FIELD OF THE INVENTION

This invention relates to the production of topical dosage forms containing human plasma fibronectin in combination with growth factors having human mitogenic or angiogenic activity, such as platelet-derived growth factor (PDGF-ββ) and keratinocyte growth factor-2 (KGF-2), to promote wound healing in humans.

### BACKGROUND OF THE INVENTION

Fibronectin is a ubiquitous extracellular glycoprotein containing around 5% carbohydrate. It exists in a soluble form in body fluids and in an insoluble form in the extracellular matrix. Fibronectin plays a major role in many important physiological processes, such as embryogenesis, hemostasis, thrombosis and wound healing (Potts and Campbell, 1994). The characteristic form of plasma fibronectin is a disulfide-bonded dimer of 440,000 daltons, each subunit having a molecular weight of about 220,000 daltons. Plasma fibronectin is also known by various other names, including cold-insoluble globulin, antigelatin factor, cell attachment protein, cell spreading factor, and opsonic alpha 2-surface binding glycoprotein. These names reflect biological activities of fibronectin such as cell recruitment, opsonization of particulate debris, and promotion of wound contraction. Reviews on structure and activities of fibronectin have been published elsewhere (Hynes, R. O., 1990).

Wound healing is usually divided into three phases: the inflammatory phase, the proliferative phase, and the remodeling phase. Soluble (growth factors and cytokines) and insoluble (extracellular matrix proteins) mediators govern these processes. Fibronectin has been reported to be involved in each stage of the wound healing process (reviewed by Brotchie and Wakefield, 1990), particularly by creating a scaffold to which the invading cells can adhere. Initially, there is a release of many mediators to the wound site such as fibronectin and fibrinogen. Fibronectin promotes inflammatory cell migration into the wound and debris phagocytosis by monocytes. Thereafter, angiogenesis and reepithelialization take place. At this stage, fibronectin exerts chemotactic activity on endothelial cells, and promotes epithelial cell and fibroblast migration onto the basal membrane. Fibronectin also appears to be an essential component of the remodeling phase where it plays a major role in the organization of collagen fibrils. The fibrillar collagen ultimately forms fibrous bundles that greatly enhance the tissue tensile strength, leading to wound closure.

A large number of growth factors are present in wounds, being released from platelets and secreted by cells migrating into the wound (Singer and Clark, 1999). These growth factors exhibit a large array of biological activities which are crucial to wound healing, as detailed by Bennett and Schultz (1993a, 1993b). These activities include recruitment of cells into the wound, stimulation of cell proliferation and stimulation of synthesis of extracellular matrix components. Platelet-derived growth factors (PDGF), fibroblast growth factors (FGF), keratinocyte growth factors (KGF-1 and KGF-2), transforming growth factor-betas (TGF-β), vascular endothelial growth factor (VEGF), granulocyte-macrophage colony-stimulating factor (GM-CSF), insulin-like growth factors (IGF-I and IGF-II) and epidermal growth factor (EGF) are among the soluble mediators which may be involved in wound repair.

As reviewed by Robson et al. (1998), chronic wounds are generally deficient in one or many of the growth factors required for healing. While the messenger RNA may be present, the growth factor itself may not be synthesized, may be trapped within fibrin cuffs surrounding capillaries or quickly destroyed by matrix metalloproteinases. For example, levels of PDGF, bFGF, EGF and TGF-β were significantly lower in fluids from chronic wounds than from acute wounds (Cooper et al., 1994). Similarly, fibronectin in chronic wounds is subjected to degradation (Grinnell et al., 1992). The application of exogenous recombinant growth factors and fibronectin to chronic wounds has thus been a promissing route to promote healing.

Topically applied plasma fibronectin has been reported as being useful for increasing the rate of wound healing such as in corneal wounds (Nishida et al., 1984; Phan et al., 1987) and leg ulcers (Wysocki et al., 1988). Various dressings containing significant amounts of fibronectin have been described in the prior art. For example, Brown and Blun disclosed methods to form mats of fibronectin fibres (U.S. Pat. Nos. 5,629,287 and 5,610,148) while Reich (U.S. Pat. No 4,973,466) describes a fibronectin gel matrix. However, these dressings do not allow the release of the fibronectin into the wound. Similarly, the application of growth factors to wounds enhance normal repair and reverse deficient repair in a number of animal models of dermal incisional and excisional wound repair (see for example Pierce et al, 1988; Greenhalgh et al., 1990; Mustoe et al., 1991; Jimenez and Rampy, 1999; Xia et al, 1999). However, results from clinical trials involving these growth factors have been less than encouraging (Robson, 1996; Robson et al., 1998).

A major limiting factor in developing an effective topical dosage form of a drug is not only having an active drug, but also having a formulation that allows the passage of the active drug from the carrier into a site of delivery. Very active drugs such as growth factors may have no therapeutic value if the topical formulation does not allow the drug to move from the semi-solid carrier into the wound. Moreover, since the healing process involves a large number of growth factors and extracellular matrix proteins, each having specific functions, the healing results from the concerted action of all the players. Therefore, it would be highly desirable to develop formulations which would contain several mediators of the repair process and which would maximize the contact time of the fibronectin/growth factors with the wound and also control their release to the wound, thereby leading to high absorption values.

Examples of solid wound dressings capable of delivering an effective wound healing amount of fibronectin to a wound site have been described in the WO0113967A1 entitled “Solid Wound Healing Formulations Containing Fibronectin”, which is incorporated herein by reference in its entirety. The present invention describes delivery systems in which effective doses of fibronectin and the growth factor PDGF-ββ are combined in solid wound healing formulations.

### SUMMARY OF THE INVENTION

The present invention provides techniques for the creation of solid wound dressings capable of delivering an effective wound healing amount of fibronectin in combination with growth factors to a wound site. Examples of solid dressings used to deliver the fibronectin/growth factors are based on calcium alginate, carboxymethylcellulose (CMC), hydroxypropylcellulose (HPC), carbomer and carrageenan.

Formulation of topical dosage forms intended for the incorporation of fibronectin and growth factors should respect several quality criteria. All components of the preparation including solvents and gelling agents should be nontoxic for the wound and compatible with the drug. The final product should promote optimal release of the drugs to its site of action, be of adequate consistency to enhance contact time of the drug with the wound and be sterile. The formulations of the present invention do not affect or change the biological activities of the fibronectin or growth factors incorporated in the formulations. The fibronectin and growth factors maintain their characteristic biological acitivities before and after their incorporation into the wound formulations of the invention.

Use of solid dressings of the present invention offer specific advantages in terms of dose reproducibility, ease of storage, transport and application. In addition, preservatives are not needed.

These solid dressings provide a slow release of fibronectin and growth factors to the delivery site. This should allow for the application of the solid wound dressings on a convenient basis, i.e. once a week to twice a week. Due to this low frequency application schedule and the solid form of the dressing, the trauma done to the wound by the removal of depleted doses should be minimized.

The preferred formulations of this invention can be used with other wound healing promoters having a composition similar to fibronectin, such as proteins of similar size (thrombospondin, laminin, vitronectin, fibrinogen) or smaller size (such as peptides including growth factors).

The preferred formulations can be evaluated using an in vitro diffusion cell system consisting of a rigid receptor containing a deepithelialized skin sample, the deepithelialized side facing upwards into a donor compartment and the dermal side facing downwards into a receptor compartment as described in detail in U.S. Pat. No. 5,877,149, entitled “Deepithelialized Skin Diffusion Cell System,” which is incorporated herein by reference in its entirety. The receptor compartment is connected to a circulating buffer circuit, with the buffer temperature maintained at 37° C., while the skin surface is about 32° C. The pharmaceutical delivery system comprising a fibronectin-polysaccharide dressing is characterized in that 80% of the fibronectin is absorbed in a dermal layer of a deepithelialized skin diffusion cell system after 12 hours.

### BRIEF DESCRIPTION OF THE FIGURES

FIG. 1 describes the histomorphometric measurements of new granulation tissue. MH refers to the maximum height at the advancing edges of the wound and GAP the remaining diameter of the wound without new granulation tissue.

FIG. 2 describes the difference of maximum height means (adjusted MHM) of new granulation tissue between control and treated groups. A: DERMALINK™ delivery system releasing 80 μg fibronectin/mm² wound surface area; B: DERMALINK™ delivery system releasing 80 μg fibronectin and 0.17 μg rhPDGF-ββ/mm² wound surface area; C: DERMALINK™ delivery system releasing 80 μg fibronectin and 0.35 μg rhPDGF-ββ/mm² wound surface area; D: DERMALINK™ delivery system releasing 0.17 μg rhPDGF-ββ/mm² wound surface area; E: DERMALINK™ delivery system releasing 0.35 μg rhPDGF-ββ/mm² wound surface area. rhPDGF-ββ refers to recombinant human PDGF-ββ. The amounts released in these and other figures are measured using the deepithelialized skin diffusion cell system as described in detail in U.S. Pat. No. 5,877,149, which is incorporated herein by reference in its entirety

FIG. 3 describes the difference of new granulation tissue volume means (adjusted NGV) between control and treated groups. A: DERMALINK™ delivery system releasing 80 μg fibronectin/mm² wound surface area; B: DERMALINK™ delivery system releasing 80 μg fibronectin and 0.17 μg rhPDGF-ββ/mm² wound surface area; C: DERMALINK™ delivery system releasing 80 μg fibronectin and 0.35 μg rhPDGF-ββ/mm² wound surface area; D: DERMALINK™ delivery system releasing 0.17 μg rhPDGF-ββ/mm² wound surface area; E: DERMALINK™ delivery system releasing 0.35 μg rhPDGF-ββ/mm² wound surface area. rhPDGF-ββ refers to recombinant human PDGF-ββ.

FIG. 4 describes the difference of new granulation tissue gap means (adjusted NGTGAP) between control and treated groups. A: DERMALINK™ delivery system releasing 80 μg fibronectin/mm² wound surface area; B: DERMALINK™ delivery system releasing 80 μg fibronectin and 0.17 μg rhPDGF-ββ/mm² wound surface area; C: DERMALINK™ delivery system releasing 80 μg fibronectin and 0.35 μg rhPDGF-ββ/mm² wound surface area; D: DERMALINK™ delivery system releasing 0.17 μg rhPDGF-ββ/mm² wound surface area; E: DERMALINK™ delivery system releasing 0.35 μg rhPDGF-ββ/mm wound surface area. rhPDGF-ββ refers to recombinant human PDGF-ββ.

### DETAILED DESCRIPTION OF THE INVENTION

The solid wound formulations, dressings and delivery systems of the present invention deliver fibronectin and one or more growth factor to a wound site in order to enhance, accelerate or improve healing.

Fibronectin, although encoded by a single gene, exists in multiple forms that differ in sequence and function due to the alternative splicing of exons corresponding to the EDA, EDB and IICS domains of the protein (Hynes, 1990). Generally, plasma fibronectin differs from cellular fibronectin in that it lacks the EDA and EDB domains. Fibronectin useful to the present invention may be the plasmatic or the cellular form, of natural or recombinant source, or may consist of combinations of recombinant functional domains. Preferably, full-length fibronectin will be used. Plasma fibronectin may be purified from plasma or plasma cryoprecipitate as described by Howoritz and Chang (1989). Recombinant fibronectin may be produced using transformed eukaryotic cells as described in patent WO9008833. Methods to produce biologically active fragments of fibronectin have been disclosed in U.S. Pat. No. 5,958,874.

In another embodiment of the present invention, other extracellular matrix proteins such as laminin, vitronectin, fibrinogen, fibrin, thrombospondin could replace fibronectin in the solid wound dressing of this invention.

The growth factors referred to herein are those having human mitogenic or angiogenic activity, selected from the group consisting of PDGF, KGF (FGF-7), KGF-2 (FGF-10), acidic FGF, basic FGF, TGF-β, IGF-I, IGF-II, VEGF and GM-CSF. It is understood that purified naturally occurring growth factors, growth factors produced by recombinant techniques or biologically active fragments thereof chemically synthesized or produced by recombinant techniques may all be used with the present invention. It is preferred that growth factors be produced by recombinant techniques for use in the present invention.

PDGFs are released by platelets at sites of tissue damage and are secreted by numerous cell types, including fibroblasts and endothelial cells. They are chemotactic for fibroblasts and stimulate the proliferation of fibroblasts and smooth muscle cells. PDGFs exist as dimers composed of two polypeptides, the α and the β chains, which are linked by disulfide bonds in three different combinations: αα, αβ and ββ. As used herein, PDGF-ββ refers to PDGF having the polypeptide sequence or any substantial portion thereof as described in Johnsson et al. (1984). The human c-sis gene and its simian sarcoma viral analog (v-sis gene) encode the precursor of the PDGF-β protein which is subsequently cleaved to produce the active PDGF-ββ. PDGF-ββ may be purified from platelets (Heldin et al., 1979; Deuel et al., 1981). Polypeptides that have biological activity similar to that exhibited by the natural human PDGF polypeptide may also be used, such as recombinant PDGF similar to the native form (see for example U.S. Pat. No. 5,516,896, which is incorporated herein by reference in its entirety; Hoppe et al., 1989) or mutants, muteins, analogs or biologically active fragments having a mitogenic activity resembling that of mature human PDGF-ββ (see for example U.S. Pat. No. 5,905,142, U.S. Pat. No. 5,187,263, U.S. Pat. No. 5,149,792, U.S. Pat. No. 5,759,815), including precursors that are transformed into active PDGF in situ by proteolytic processing.

In order to obtain a solid dressing or delivery system which can be easily handled without breaking, and which can be applied to a wound without causing discomfort due to high salt concentrations, it is necessary to solubilize in water the components included in the dressing. An effective wound healing amount of fibronectin for use in the present invention may be within the range of about 30 to 160 μg/mm². It is prefered that fibronectin concentration be about 35 to 90 μg/mm² and most prefered about 80 μg/mm². An effective wound healing amount of a growth factor for use in combination with fibronectin in the present invention will depend in part of the growth factor selected and its potency. The PDGF-ββ concentration may be within the range of about 0.1 to 1 μg/mm². It is prefered that PDGF-ββ concentration be about 0.15 to 0.6 μg/mm² and most prefered about 0.35 μg/mm². More than one growth factor may be concomitantly present in the solid dressing containing fibronectin, and in that case, each growth factor will be present at its own effective concentration.

The extracellular matix proteins and growth factors in the solid wound formulations, dressings and delivery systems of the present invention retain their structural and functional integrity, which are characteristic and identify the proteins and growth factors. For example, fibronectin retains characteristic biological activities such as gelatin-binding, heparin-binding and cell-adhesion activities.

The solid wound formulations, dressings and delivery systems of the present invention also contain a plant polysaccharide which may be alginates, carrageenans, cellulose derivatives such as carboxymethyl-cellulose (CMC), hydroxypropylcellulose (HPC) or oxidized cellulose derivatives. In its preferred embodiment, the plant polysaccharide consists of calcium alginate fibers, obtained by a freeze-drying process. Alginates are naturally occurring substances extracted from marine brown algae and used in the pharmaceutical, cosmetic, textile and food industry. Alginates are polyanionic polysaccharides composed of linear binary copolymers of D-mannuronic acid and L-guluronic acid. The most common uses are based on the polyelectrolytic nature of the alginates, which provides the basis of their gelling properties and their ability to swell. The commercially available sodium alginates are water soluble. When such alginates are added to a solution containing polyvalent ions, for example bivalent alkaline earth metal ions such as Ca⁺⁺, alginate gels having a semi-solid form are produced. This is a result of a ionic crosslinking of several alginate chains.

Calcium alginates have long been known for their ability to form fibres or nonwoven materials. These have been used primarily as swabs or dressings for medical, surgical or other purposes, such as described in European Patent Specification, EP 0721355 B1, entitled “Alginate Wound Dressings”, which is incorporated herein by reference in its entirety. Supplied in the form of nonwoven wound dressings for the treatment of exudating wounds, the calcium alginate dressing is said to encourage the formation of controlled ion-active gel over the wound site which reacts with the sodium ions in the exudate. Examples of exudative wounds include pressure ulcers, venous stasis ulcers, diabetic ulcers, arterial ulcers, second degree bums and skin graft donor sites. When prepared according to the present invention, the solid calcium-alginate-fibronectin-PDGF-ββ dressing placed onto an exudating wound turns into a gel-like state which allows the fibronectin and PDGF-ββ to move freely from the gel into the wound. This allows the controlled release of the therapeutic components into the wound and high absorption values. In one embodiment of the pharmaceutical delivery system according to the present invention is a fibronectin-polysaccharide dressing wherein at least 80% of the fibronectin is absorbed in a dermal layer of a deepithelialized skin diffusion cell system after 12 hours.

The following examples illustrate specific embodiments of the present invention. The invention is not be considered limited by these examples, but only by the appended claims.

### EXAMPLE 1

### Solid Fibronectin-rhPDGF-ββ Calcium-Alginate Wound Dressing Formulation

Alginate salts can be converted into fibers by a process of freeze-drying. This procedure produces a sponge-like structures with hydrophilic properties. In the presence of fluids, the dressings turn into a gel-like state, capable of absorbing up to 20 times their weight in wound exudate. The fibrous gel thereby creates the desired moist environment for the wound. The dressings can also be removed with a minimal amount of discomfort, and granulating tissue and epithelial cells are not traumatized during dressing changes. Calcium-alginate dressings in particular are recommended for use on exuding wounds, such as pressure ulcers, venous stasis ulcers, diabetic ulcers, arterial ulcers, second degree burns and skin graft donor sites.

The fibronectin/recombinant human PDGF-ββ (rhPDGF-ββ) calcium-alginate wound dressings according to the present invention deliver a high concentration of fibronectin along with effective wound healing doses of rhPDGF-ββ into the wound site.

The basic mechanisms at play for the fibronectin/rhPDGF-ββ-calcium alginate dressing is that when this dressing comes into contact with the sodium in the exudate, ion exchange occurs, turning the calcium alginate fibers into a protective non-adherent film gel. In this gel state, fibronectin and rhPDGF-ββ are free to move from the gel into the wound.

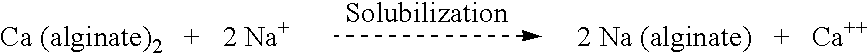

Ca⁺⁺ forms an insoluble alginate salt and Na⁺ forms a soluble alginate salt (the equivalent ratio of the first to second cations being 50:50, here 0.2 M NaCl and 0.2 M CaCl₂). The maximum homogeneity in the dressing is reached by an appropriate concentration of both gelling and non-gelling cations. Additional Na⁺ comes from the exudate or, if the wound is too dry or there is no exudate, a small amount of saline can be added to the wound immediately prior to placing the dressing.

Because alginates are anionic polysaccharides, the complex is preferably formed by combining the fibronectin, the rhPDGF-ββ, and sodium alginate at a pH which is no higher than the isoelectric point of the proteins (pI 6.2 for fibronectin and pI 9.2 for rhPDGF-ββ) where they are positively charged. This pH is obtained by adding glacial acetic acid for a final pH around 5.0. Since acetic acid is highly volatile, a certain amount of acetic acid is removed during the freeze-drying process which is under a vacuum. The final pH of the dressing is around 6.5.

The mixed salt alginate dressing exhibits a highly effective combination of properties. For example, there is enough insolubilizing cation in the mixed salt alginate to make the product relatively easy to manipulate. There is also enough solubilizing cation to facilitate the release of fibronectin and rhPDGF-ββ into the wound. The combination of soluble and insoluble alginate fibers has the further advantage that the dressing is both easily removed after the wound treatment and easily applied initially.

Fibronectin/rhPDGF-ββ Calcium-Alginate Dressing Composition

Three commercially available preparations of sodium alginate were tested. Protanal LF 120 M sodium alginate (Pronova Biopolymer, Inc., Drammen, Norway) yielded a product more brittle than the preferred embodiment described below. In addition, this sodium alginate yielded a placebo dressing (i.e., control without fibronectin) which was yellowish in appearance compared to the same dressing containing fibronectin. Consequently, this formulation could not be used in human clinical trials. Pronatal LF 10/60 (Pronova Biopolymer, Inc., Drammen, Norway) produced dressings of adequate appearance and of suitable flexibility. However, due to significantly high endotoxin levels in this alginate, Pronova UP LVG sodium alginate (Pronova Biomedical A.S., Oslo, Norway) was preferred.

In a previous study, we determined that the optimal effective concentration of fibronectin was 80 μg/mm², which produced a fibronectin-calcium-alginate dressing designated DERMALINK™ delivery system (Beaulieu et al., WO 0113967). In one embodiment of the present invention, the DERMALINK™ delivery system containing various amounts of rhPDGF-ββ is prepared as follows: 1 g of sodium alginate (Pronova UP LVG, Pronova Biomedical A.S., Oslo, Norway) is dispersed in 99 g of tap water demineralized using Millipore Milli-Q water system (MQW) containing 150 μL of glacial acetic acid with a paddle type stirrer for about 1 hour at 50° C. This dispersion provides 100 mL of alginate solution 1% (w/w) at pH 4.2 which is then sterilized by filtration under 0.22 μm PVDF filter (Millex-GV, Millipore Corp., Bedford, Mass.). The pH of 10 mL MQW is adjusted at pH 11.6 with the addition of 23 μL of NaOH 3M. The lyophilized fibronectin (0.1 g) is next dissolved in MQW pH 11.6 under gentle agitation. The solution is maintained at 37° C. until complete solubilization of fibronectin occurs. This solution is then filtered through a 0.22 μm PVDF filter (Millex-GV, Millipore Corp., Bedford, Mass.). The lyophilized rhPDGF-ββ is first dissolved in MQW at a fixed concentration of 1 mg/mL and then filter sterilized as for the fibronectin solution. The 10 mg/ml sterile solution of fibronectin (3.4 ml), the 1 mg/ml rhPDGF-ββ solution (73.5 to 147 μl) and the sterile-filtered 1% solution sodium alginate (1.6 mL; pH 4.2) are then mixed into syringes. Care is taken to avoid introduction of air bubbles. Contamination is avoided by working in an aseptic environment, such as under a laminar flow hood. Generally, two syringes are used, and multiple exchanges under pressure are applied. An adapter device, such as a female luer connection can be used to connect the syringes or other exchange devices. Vigorous agitation is minimized in order to avoid fibronectin precipitation.

Gellation of the solution is achieved by the addition of 15 μL 0.2 M NaCl+0.2 M CaCl₂. At this point, the fibronectin-rhPDGF-ββ calcium alginate complex forms an emulsion-like solution at a pH between 5.0 and 5.2. This solution is then placed in a borosilicate glass vial (5 mL for a surface area of 5.3 cm²) and frozen at −20° C. for 2 hours and 30 minutes at −80° C. The water is then removed by freeze-drying using a Labconco freeze-dryer (model 77580, Kansas City, Mo.). By this technique, a 4.2 cm² solid sponge-like fibronectin-rhPDGF-ββ calcium alginate delivery system (DERMALINK™ delivery system containing rhPDGF-ββ) with a fibronectin concentration up to 80 μg/mm² and with a rhPDGF-ββ concentration ranging from 0.17 to 0.35 μg/mm² . In this particular embodiment, the delivery system is produced in a disk shape. The delivery system contains up to about 0.27 to about 0.32% PDGF by weight.

The DERMALINK™ delivery system disk containing rhPDGF-ββ can be readily removed from the vial by creating a moistened environment which is provided by plugging the opening of the vial with a wet gauze for 10 minutes. The disk becomes soft and flexible, and can be removed using tweezers.

### EXAMPLE 2

### Solvent/Detergent-Treated Human Homologous Plasma Fibronectin

The lyophilized fibronectin used in Example 1 was prepared as described in this example. Fibronectin may be isolated from plasma cryoprecipitate purchased from licensed manufacturers of human blood plasma products or isolated from whole plasma. First, lots of cryoprecipitate or lots of plasma prepared from different donors are tested for atypical antibodies, hepatitis B and C virus (HBV, HCV), human immunodeficiency virus (HIV), human T-cell lymphotrophic virus (HTLV), cytomegalovirus (CMV) and syphilis. Second, a viral inactivation by the solvent/detergent method using tri(n-butyl)phosphate (TNBP) and Triton X-100 is performed. This treatment of plasma was shown to inactivate very large quantities of HBV, HCV and HIV (Horowitz et al., 1992) without affecting labile proteins such as fibronectin.

Cryoprecipitate is dissolved in 50 mM Tris-HCl pH 7.5+150 mM NaCl for 5 hours at 37° C. The solution is then clarified by sequential filtration through 1)1.5% Celite (a filter aid; Van Waters and Rogers Ltd., Vancouver, British Columbia) and 30 LP CUNO depth filters (Peacock, Lasalle, Quebec), 2) an 8 μm filter (Sartopure-PP2-membrane filter, Sartorius Corp., Edgework, N.Y.) and 3) a 1.2 μm filter filter (Sartopure-PP2-membrane filter, Sartorius Corp., Edgework, N.Y.). The cryoprecipitate is then treated for viral inactivation. The clarified cryoprecipitate is stirred for 21 hours at 24° C. in the presence of 0.3% (vol/vol) TNBP and 1% (vol/vol) Triton X-100. Alternatively, plasma from several donors is thawed and used as the source of fibronectin. Thawed plasma is stired for 6 hours at 24° C. in the presence of 1% (vol/vol) TNBP and 1% (vol/vol) Triton X-100. Soybean oil (commercially available, food grade) (20% vol/vol) is then added, mixed gently for 30 minutes at ambient temperature and then removed by decantation in a funnel, at 4° C.

Fibronectin is isolated from the solvent/detergent-treated human plasma or cryoprecipitate using a gelatin-Sepharose affinity chromatography procedure (Horowitz and Chang, 1989). This method takes advantage of the affinity of fibronectin for gelatin in a procedure that allows isolation of electrophoretically pure fibronectin from human plasma with excellent yields. In this method, gelatin is covalently coupled to Sepharose CL-4B (Amersham Pharmacia Biotech, Baie d'Urfé, Québec) after CNBr activation. The binding capacity for human plasma fibronectin provided by this system is >1 mg/mL of gel. The purificaton is performed in a batch procedure with a glass funnel filter holder (Costar Nucleopore, Pleasanton, Calif.) with a capacity of 375 mL and a filtration area of 10.5 cm² at a flow rate of 25 mL/min. Briefly, the plasma sample is passed twice on a gelatin-Sepharose gel (Amersham Pharmacia Biotech, Uppsala, Sweden). The matrix is washed with three column volumes of 50 mM Tris-HCl buffer pH 7.5+150 mM NaCl, three column volumes of 50 mM Tris-HCl buffer pH 7.5+1 M NaCl and again with three column volumes of 50 mM Tris-HCl buffer pH 7.5+150 mM NaCl. Elution is carried out with 4 column volumes 0.1 M glycine pH 11.0. The resulting solution of fibronectin is then concentrated about 5 times by ultrafiltration under nitrogen then diafiltered with 4-5 volumes of water (quality for injection) or until the final conductivity of the fibronectin solution is about 600 μS/cm, using a standard Flex Stand™ Bench Top Pilot System equipped with a size 8 ultrafiltration cartridge (100 K membrane, 1 mm hollow fibres) (A/G Technology Corp., Needham, Mass.). The pH of the purified fibronectin solution is adjusted to 11.0 with 1 N NaOH and the solution concentrated to 10 mg/ml prior to the preparation of dressings as described in Example 1. Alternatively, the fibronectin may be lyophilized and frozen at −80° C. until needed.

The purified fibronectin solution is verified for contamination by TNBP and Triton X-100. TNBP is quantified in a sample of purified fibronectin after hexane extraction by gas chromatography using a 6 mm by 2 mm internal diameter (ID) by 1.2 m glass column packed with 10% Sephacryl SP-1000 (Amersham Pharmacia Biotech, Baie d'Urfé, Québec) on a 80/100 mesh Supelcoport (Supelco, Bellafonte, Pa.). Triton X-100 was assayed by injecting a sample of purified fibronectin to high pressure liquid chromatography (HPLC) gel filtration column G2000 SW 7.5 mm ID by 60 cm (Tosohass; Tosoh Biosep, Montgomeryville, Pa.) coupled with a UV detector set at 230 nm. Fibronectin preparations were found to contain less that 1 ppm of either TNBP or Triton X-100.

The protein concentration is determined using the Bio-Rad protein assay (Bio-Rad Laboratories, Richmond, Calif.).

Fibronectin from non-human animals can be purified using similar methods. The lots of plasma would be screened for atypical antibodies as appropriate for the source organism and known to those skilled in the art. For example, domestic cat plasma would be screened for feline leukemia virus.

### EXAMPLE 3

### Production of Recombinant Human PDGF-ββ (rhPDGF-ββ)

Several methods have been established for the production of rhPDGF-ββ or its functional analogs. Murray et al. (1988) describes its production in eukaryotic cells (U.S. Pat. No. 4,766,073, which incorporated herein by reference). Expression in bacterial cells as been the subject of several publications including those of Lyons et al. (1995) (U.S. Pat. No. 5,428,135, which is incorporated herein by reference) and Alexander et al. (1992) and could be used instead of the one described below.

Construction of the PDGF Expression System

Two oligonucleotides (5′ CGCGGTACATATGAGCCTGGGTTCCCTGACCATTGCT and 5′ GCGGATCCCTATTAGGTCACAGGCCGTGCAGCTGC) were designed to amplify the sequence coding for the mature form of human PDGF-β. Primers were synthesized by the “service de synthése d'ADN et d'analyse d'image” (Centre de recherche du CHUL, Ste-Foy, Québec). The PDGF-D sequence (nt 361-687 in GenBank accession #X02744) was amplified using the plasmid pSM-1 (ATCC clone #57050) as a template under the following polymerase chain reaction (PCR) conditions: 5 cycles at 94° C. for 1 min, 59° C. for 1 min., 72° C. for 30 sec. followed by 20 cycles at 94° C. for 1 min., 64° C. for 1 min., 72° C. for 30 sec. using Taq polymerase (Amersham Pharmacia Biotech, Baie d'Urf\acute{e+EE, Qu\acute{e}}bec), in a MJ Research PTC-100 thermocycler (Washington, Mass.). The resulting PCR product has a NdeI site at its 5′ end, providing a methionine codon in-frame with the sequence coding for the mature form of PDGF-β which will serve as the translation initiation site for recombinant expression in E. coli. It also has two in-frame stop codons and a BamHI site at its 3′ end. The PCR product was digested with the appropriate restriction enzymes and cloned in the corresponding sites of the vector pET-11a (Novagen Inc., Madison, Wis.). The resulting recombinant vector was designated pETPD. The E. coli strain BL21(DE3) (Novagen Inc., Madison, Wis.) was transformed with pETPD to produce the recombinant PDGF-β expression system.

Cell Growth, Expression of rhPDGF-β and Purification of Inclusion Bodies

The E. coli strain containing pETPD was grown at 37° C. in Luria-Bertani broth in the presence of 50 μg/ml ampicillin on a rotary shaker set at 200 rpm until an OD_{600 nm} of 0.6-0.9 (preferably 0.8) was reached. Isopropyl-β-D-galactopyranoside (IPTG) was then added to the culture to a final concentration of 0.5 mM. The culture was incubated for three additional hours at 37° C. Bacterial cells were collected by centrifugation at 5000×g for 20 min., the growth medium was discarded and cell pellets were frozen at −80° C.

The cell paste was thawed and resuspended in about 1/30 culture volume of TE (20 mM Tris-HCl, 1 mM EDTA, pH 8.0). Cells were lysed by two passages in a FRENCH® pressure cell press (SLM Aminco, SLM Instruments Inc., Urbana, Ill.) set at a pressure of 14000-16000 psi. The lysate was centrifuged at 15 000×g, at 4° C., for 20 min. The pellet, consisting principally of rhPDGF-β inclusion bodies (IBs), was resuspended in 1/30 culture volume of TE containing 2% Triton X- 100, left under gentle rocking agitation for 10 min. at room temperature and centrifuged as above. IBs were then resuspended in a similar volume of TE containing 2% Triton X-100 and 2.5 M urea, left under gentle rocking agitation for 10 min. at room temperature then centrifuged as above. IBs were rinsed once in TE and subsequently stored at −80° C. until the renaturation of rhPDGF-ββ.

Refolding of rhPDGF-ββ from Inclusion Bodies

The refolding procedure is a modification from the method described by Alexander et al. (1992). IBs were resuspended in about 1/50^{th} culture volume of 6 M guanidium-HCl (GdCl). Dithiothreitol (DTT) was added to a final concentration of 5 mM. The mixture was incubated under gentle agitation at room temperature for one to two hours. The solubilized rhPDGF-β was centrifuged at 48 000×g, for 1 hour at 20° C. and the supernatant was diluted with 6 M GdCl to adjust the protein concentration to approximately 2.4 mg/ml. The pH of the protein solution was adjusted to 7.5 with NaOH. Solid sodium sulfite and sodium tetrathionate were added to a final concentrations of 111 and 13 mM respectively and proteins were sulfonated for 4 hours at room temperature while mixing. Sulfonated proteins were dialyzed against several changes of buffer A (50 mM Tris-HCl, pH 7.8, 2 mM EDTA, 8 M urea, 5 mM reduced glutathione, 0.5 mM oxidized glutathione). The dimerization/renaturation was then allowed to proceed by a “continuous-flow” dialysis method: The dialysis bag was placed in a volume of buffer A. An equal volume of buffer A lacking urea was then pumped into the dialysis bath, with mixing, at a rate equal to that at which the contents of the diluted bath were being pumped out. This gradient was continued over about 72 hours at room temperature. At this stage, the dialysis bag contains precipitated, denatured rhPDGF-β, and a mixture of soluble rhPDGF-β (monomers) and rhPDGF-ββ (dimers) in a proportion of approximately 1:3. This mixture was dialyzed against buffer B (20 mM Tris-HCl, pH 7.5), concentrated about 3 times using an Amicon ultrafiltration unit (Millipore Corp., Bedford, Mass.) equipped with an Amicon Ultracel-YM-10 membrane (Millipore Corp., Bedford, Mass.). The rhPDGF-ββ (dimer) was purified from rhPDGF-β (monomer) on a SP-sepharose HiTrap column (1 ml bed volume; Amersham Pharmacia Biotech, Baie d'Urf\acute{e+EE, Qu\acute{e}}bec). Proteins were loaded on the column in buffer B. The column was washed with approximately 5 column volumes of buffer B. Elution was achieved in steps. Monomers were eluted in 0.3 M NaCl in buffer B while dimers were eluted in 0.7 M NaCl. Dimers were dialyzed against MQW, frozen in 6-ml vials and lyophilized.

The purified rhPDGF-ββ is about 95% pure as determined by SDS-PAGE and Coomassie Blue staining. Yields were about 2.5 mg rhPDGF-ββ per liter of recombinant E. coli culture.

### EXAMPLE 4

### Comparative Study of DERMALINK™ Delivery System and DERMALINK™ Delivery System Containing Various Amounts of rhPDGF-ββ in Promoting the Healing of Rabbit Ear Dermal Ulcers

The Rabbit Ear Dermal Ulcer Model

The study of the efficacy of DERMALINK™ delivery system and DERMALINK™ delivery system containing various amounts of rhPDGF-ββ in stimulating wound healing was performed using the rabbit ear dermal ulcer model of wound healing as developed by Mustoe et al. (1991).

Young adult New Zealand white female rabbits, 3.0-3.5 kg (Charles River Laboratories, Canada) were anesthetized with ketamine (60 mg/kg) and xylazine (5 mg/kg). Using a 6-mm trephine and microsurgical instruments, four circular full-thickness 6 -mm diameter ulcers were made to the depth of bare cartilage under sterile conditions. Each wound was covered with a 6 mm-diameter piece of solid dressing immediately after surgery. Wounds were then covered with an occlusive polyurethane film (Tegaderm film, 3M, Minneapolis, Minn.) to prevent wound desiccation. Neck collars were placed on rabbits for the duration of the experiment.

The delivery systems tested are specified below. Fibronectin used to produce these dressings was prepared from frozen, whole plasma, collected from healthy volunteers among the employees of Le Centre Hospitalier de l'Université Laval, Ste-Foy, Québec, Canada. Placebo consisted of 0.7% calcium alginate dressing containing neither fibronectin nor rhPDGF-ββ.

Differences in rates of healing between treatment groups were measured 8 days following surgery. At the time of sacrifice, the ulcers were bisected and fixed in 10% buffered formalin. The specimens were then dehydrated in graded alcohol and xylene, embedded in paraffin and sectioned, taking care to obtain a cross section as near as possible to the center of the wound.

Histomorphometric Measurement of New Granulation Tissue

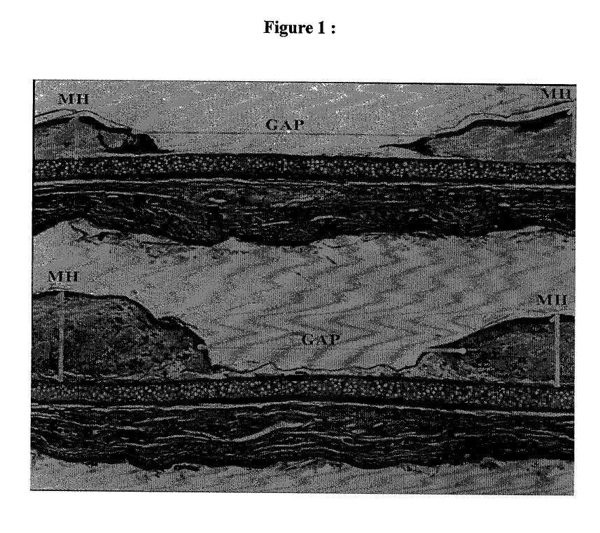

After Masson-trichrome staining of 3-μm thick sections, the new granulation tissue gap (GAP) (defined as the remaining diameter of the wound without new granulation tissue) and the maximum height (MH) of the new granulation tissue at the advancing edges of the wound (FIG. 1) were measured by histomorphometry using Biometric Bioquant true color laser vision (R&M, Nashville, Tenn.). Each MH value represents the mean of four measurements of the maximum height of the new granulation tissue on each side of an ulcer, for two tissue sections for each ulcer. The surface area of the wound is calculated by the equation (GAP/2)²π. On the day of surgery (day 0), the measured GAP was 6.2 mm and the calculated surface area was 30.19 mm². To determine the new granulation tissue surface area, the area of the wound at day 8 was subtracted from the area of the wound at day 0: (day 0 GAP/2)²π−(day 8 GAP/2)²π. The new granulation volume (NGV) consists in new granulation surface area x MH. MH, NGV and new granulation tissue GAP (NGTGAP) were the three response variables used to statistically compare the treatments. Area and volume measurements for new granulation tissue were calculated based on the assumptions that the wounds healed concentrically and did not contract. Statistical analysis was carried out using a Student's paired t test for each formulations studied using Excel version 5.0 (Microsoft Corporation). All comparisons were made to paired control wounds. P<0.05 was considered significant.

Six treatments were studied:

A: DERMALINK™ delivery system releasing 80 μg fibronectin/mm² wound surface area

B: DERMALINK™ delivery system releasing 80 μg fibronectin and 0.17 μg rhPDGF-ββ/mm² wound surface area

C: DERMALINK™ delivery system releasing 80 μg fibronectin and 0.35 μg rhPDGF-ββ/mm² wound surface area

D: DERMALINK™ delivery system releasing 0.17 μg rhPDGF-ββ/mm² wound surface area

E: DERMALINK™ delivery system releasing 0.35 μg rhPDGF-ββ/mm² wound surface area

Control: DERMALINK™ delivery system releasing neither fibronectin nor rhPDGF-ββ

Four 6 mm ulcers were made on each rabbit ear. The left rabbit ears were treated with one of the five treatments previously cited (A to E) while the right ears were treated with the control.

The determination of a significant effect was first obtained by comparing the results from both ears of the rabbits. Student t-tests were performed to determine if the left ears associated to one of the five treatments showed better improvement than the right ears treated with the control.

Table 1 presents the mean differences for each treatment and response variable.

TABLE 1

| | | Difference between treatment and | | |
|---|---|---|---|---|
| | | control | | |
| | Number of | | NGV | NGTGAP |
| Treatment | rabbits | MH means | means | means |
| A | 15 | 0.08** | 3.01** | −0.416 |
| B | 8 | 0.15** | 4.19* | −0.178 |
| C | 14 | 0.25** | 11.04** | −1.368** |
| D | 8 | 0.11 | 3.61** | −0.293 |
| E | 14 | 0.14** | 5.63** | −0.435 |

*Significant at the 5% significance level, **Significant at the 1% significance level

In Table 1, a positive difference for MH and NGV indicates that the treatment is better than control while for NGTGAP, a negative difference is associated to a treatment better than control. The differences marked with an asterisk are associated with statistically significant treatment effect. The treatment C is the only treatment with an effect significantly better than the control for all three response variables.

These first results compare each treatment to the control. The following analyses compare the five treatments and test if these treatments produce significantly different effects. These tests allow to determine the best treatment among those producing an improvement in comparison to the control. In order to adjust for the intrinsic healing properties of the rabbits, the differences between the measures on both ears are used as response variables. The fact that the control treatment was used on all rabbits allows to use it as a control and to have a response for the treatments not influenced by the rabbits characteristics.

The adjusted treatment values (indiced adj) are obtained by substracting the value of the control ear (right ear indiced R) from the value of the treated ear (left ear indiced L)

MH_{adj}=MH_{L}−MH_{R}

NGV_{adj}=NGV_{L}−NGV_{R}

NGTGAP_{adj}=NGTGAP_{L}−NGTGAP_{R}

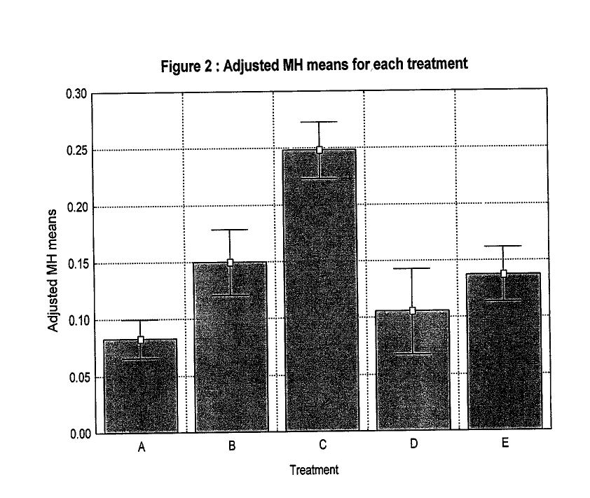

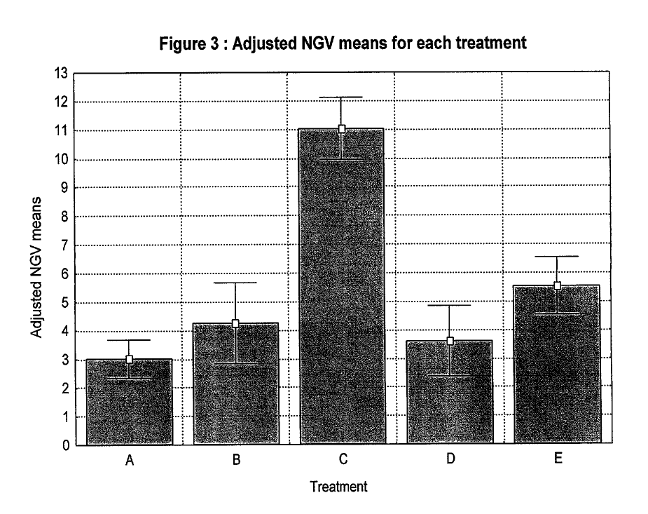

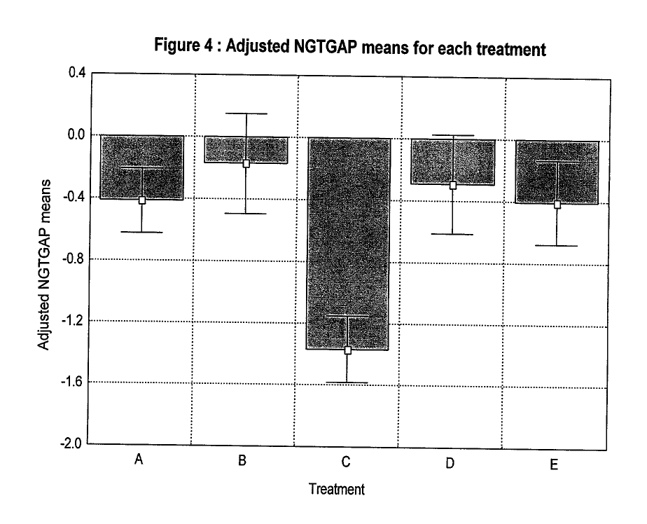

When the five treatments are compared for the three variables (FIGS. 2, 3, 4), the tests conclude that the effect of treatment C is significantly better than the other treatments at the following significance levels: MH (p=0.0058), NGV (p<0.0001) and NGTGAP (p=0.05).

When fibronectin and rhPDGF-ββ are delivered concomitantly into an acute wound, a potent wound healing promoting activity is observed. This activity leads to accelerated a wound closure. This is not observed when each agent is administered individually.

Combination therapy with rhPDGF-ββ and fibronectin, administered with an efficient delivery system such as DERMALINK™ delivery system, could prove to be clinically superior than presently available therapeutic modalities in treating chronic wounds such as venous stasis and diabetic foot ulcers.

### EXAMPLE 5

### Recombinant Fibronectin

Human or veterinary fibronectin produced by recombinant means may be utilized in the solid wound dressings of the invention in place of the plasma fibronectin purified and sterilized according to the methods described in the previous examples. Active fragments of fibronectin or modified fibronectin fragments may be utilized in alternative embodiments of the invention, using methods well known in the art. Such methods include recombinant DNA methods and chemical synthesis methods for production of proteins. For example, recombinant fibronectin polypeptide fragments can be made in bacteria or chemically synthesized. Fibronectin, fibronectin polypeptide fragments or any polypeptide compound used in the invention can be isolated from animal tissue or plasma or produced and isolated from cell culture. They may be produced and isolated from genetically altered animals, such as transgenic animals, to generate more endogenous or exogenous forms of fibronectin. Sequences of fibronectin are known to one skilled in the art, for example, as in Komblihtt et al. (1985), incorporated herein by reference in its entirety, or, are available from Genbank, NCBI, NIH, and easily searchable, on the internet at http://www.ncbi.nlm.nih.gov. Publicly available databases are incorporated herein by reference in their entirety.

Representative examples of fibronectin fragments are disclosed in: U.S. Pat. No. 5,453,489, entitled “Polypeptide fragments of fibronectin which can modulate extracellular matrix assembly”; U.S. Pat. No. 5,958,874, entitled “Recombinant fibronectin-based extracellular matrix for wound healing”; and U.S. Pat. No. 5,922,676, entitled “Methods of inhibiting cancer by using superfibronectin”, all of which are incorporated herein by reference in their entirety. Recombinant fibronectin fragments are also available from Takara Shuzo (Otsu, Japan).

Other solid dressings, such as those described in WO 01/13967 A1, entitled “Solid Wound Healing Formulations Containing Fibronectin”, can be used as a delivery system as described for fibronectin and rhPDGF-ββ and are described in the following examples.

### EXAMPLE 6

### Solid Carboxymethylcellulose (CMC) Dressing

A preferred grade is GPR® CMC (BDH Laboratories, Ville St-Laurent, Canada). A solid wound dressing containing about (w/w) 62.2% fibronectin, 0.13-0.27% rhPDGF-ββ, 37.5% CMC is prepared as follows. CMC powder is first sterilized by using a dry-heat sterilization process at 121° C. for 30 minutes using an American Sterilizer 2020 Vacamtic Eagle series autoclave (Steris Corp., Ohio). 6 grams of CMC are dispersed in 94 mL of demineralized water and mixed with a paddle type stirrer for about 3 hours. This provides a sterile concentrated hydrogel base (6% w/w).

Separately, 50 mg of lyophilized human plasma fibronectin, prepared according to Example 2, are dissolved in deionized water (5 mL) containing 12 μL of NaOH 3M, for a final pH of 11.6. This fibronectin solution is maintained at 37° C. until the complete solubilization of fibronectin occurs. This stock solution of fibronectin (10 mg/mL) is filtered through a 0.22 μm PVDF filter (Millex-GV, Millipore Corp., Bedford, Mass.).

Lyophilized rhPDGF-ββ, prepared according to Example 3, is dissolved in MQW to a concentration of 1 mg/ml. The 10 mg/ml sterile solution of fibronectin (3.4 ml), the 1 mg/ml rhPDGF-ββ solution (73.5 to 147 μl) and the sterile concentrated CMC base (0.34 g) are then mixed with syringes as described in Example 1. The pH is adjusted to 7.0 with the addition of 25 μL HCl 1N. This solution is then placed in a borosilicate glass vial (5 mL for a surface area of 5.3 cm²) and frozen at −20° C. for 2 hours and 30 minutes at −80° C. The water is then removed by freeze-drying using a Labconco freeze-dryer (model 77580, Kansas City, Mo.). By this technique, a 4.2 cm² fibronectin-PDGF-ββ-CMC wound dressing with a sponge-like structure is produced, with a concentration of up to 80 μg/mm² fibronectin and with a rhPDGF-ββ concentration ranging from 0.17 to 0.35 μg/mm².

### EXAMPLE 7

### Solid Hydroxypropylcellulose (HPC) Dressing

Solid hydroxypropylcellulose (HPC) dressing is prepared using the preferred grade of Klucel-HF® HPC (Aqualon, Houston, Tex.). A solid wound dressing containing about (w/w) 45.4% fibronectin, 0.1-0.2% rhPDGF-ββ, 54.4% HPC is prepared as follows. HPC powder is first sterilized by using a dry-heat sterilization process at 121° C. for 30 minutes using an American Sterilizer 2020 Vacamtic Eagle series autoclave (Steris Corp., Ohio). HPC (6 g) is then dispersed in 94 mL of deionized water and mixed with a paddle type stirrer for about 3 hours. This provides a sterile, concentrated hydrogel base (6% w/w).

Separately, 50 mg of lyophilized human plasma fibronectin, prepared according to Example 2, are dissolved in 5 mL of deionized water containing 12 μL of NaOH 3M, pH 11.6. The solution is maintained at 37° C. until complete solubilization of fibronectin occurred. This stock solution of fibronectin, 10 mg/mL, is filtered through a 0.22 μm PVDF filter (Millex-GV, Millipore Corp., Bedford, Mass.). Lyophilized rhPDGF-ββ, prepared according to Example 3, is dissolved in MQW to a concentration of 1 mg/ml. The 10 mg/ml sterile solution of fibronectin (3.4 ml), the 1 mg/ml rhPDGF-ββ solution (73.5 to 147 μl) and the sterile concentrated HPC base (0.68 g) is then mixed with syringes as described in Example 1. The pH is adjusted to 7.0 with the addition of 25 μL HCl 1N. This solution is then placed in a borosilicate glass vial (5 mL for a surface area of 5.3 cm²) and frozen at −20° C. for 2 hours and 30 minutes at −80° C. The water is then removed by freeze-drying using a Labconco freeze-dryer (model 77580, Kansas City, Mo.). By this technique, a 4.2 cm² fibronectin-PDGF-ββ-HPC wound dressing with a sponge-like structure is produced, with a concentration of up to 80 μg/mm² fibronectin and with a rhPDGF-ββ concentration ranging from 0.17 to 0.35 μg/mm².

### EXAMPLE 8

### Solid Carbomer Dressing

A solid carbomer dressing is prepared using Carbopol® 974P NF carbomer (BF Goodrich, Cleveland, Ohio) as the preferred grade. A solid wound dressing containing about (w/w) 75% fibronectin, 0.15-0.3% rhPDGF-ββ, 25% carbomer was prepared as follows. 2.80 g of carbomer were dispersed in 97.2 mL of demineralized water and mixed with a paddle type stirrer for about 3 hours. This dispersion is then autoclaved to provide a sterile concentrated hydrogel base (2.80% w/w).

Separately, 50 mg of lyophilized human plasma fibronectin, prepared according to Example 2, are dissolved in 5mL of deionized water containing 12 μL of NaOH 3M, pH 11.6. The solution is maintained at 37° C until complete solubilization of fibronectin occurs. This stock solution of fibronectin (10 mg/mL) is filtered through a 0.22 μm PVDF filter (Millex-GV, Millipore Corp., Bedford, Mass.). Lyophilized rhPDGF-ββ, prepared according to Example 3, is dissolved in MQW to a concentration of 1 mg/ml. The 10 mg/ml sterile solution of fibronectin (3.4 ml), the 1 mg/ml rhPDGF-ββ solution (73.5 to 147 μl), the sterile concentrated carbomer base (0.4 g) and the necessary amount of the gelifying promoter (25 μL NaOH 3M) are mixed with syringes as described in Example 1. This fibronectin-PDGF-carbomer hydrogel is then placed in a borosilicate glass vial (5 mL for a surface area of 5.3 cm²) and frozen at −20° C. for 2 hours and 30 minutes at −80° C. The water is then removed by freeze-drying using a Labconco freeze-dryer (model 77580, Kansas City, Mo.). By this technique, a 4.2 cm² fibronectin-PDGF-ββ-carbomer wound dressing with a sponge-like structure is produced, with a concentration of up to 80 μg/mm² fibronectin and with a rhPDGF-ββ concentration ranging from 0.17 to 0.35 μg/mm².

### EXAMPLE 9

### Solid Carrageenan Dressing

A solid carrageenan dressing is prepared using Gelcarin® NF carrageenan (FMC Corporation Pharmaceutical Division, Newark, Del.) as the preferred grade. A solid wound dressing containing about (w/w) 72.9% fibronectin, 0.16-0.32% rhPDGF-ββ, 26.8% carbomer is prepared as follows. 2.50 g of carrageenan is dispersed in 97.5 mL of deionized water and allowed to be mixed with a paddle type stirrer for about 3 hours. This dispersion is then autoclaved to provide a sterile concentrated hydrogel base (2.50% w/w).

Separately, 50 mg of lyophilized human plasma fibronectin, prepared according to Example 2, are dissolved in 5 mL of deionized water containing 12 μL of NaOH 3M, pH 11.6. The solution is maintained at 37° C. until complete solubilization of fibronectin occurs. This stock solution of fibronectin (10 mg/mL) is filtered through a 0.22 μm PVDF filter (Millex-GV, Millipore Corp., Bedford, Mass.). Lyophilized rhPDGF-ββ, prepared according to Example 3, is dissolved in MQW to a concentration of 1 mg/ml. The 10 mg/ml sterile solution of fibronectin (3.4 ml), the 1 mg/ml rhPDGF-ββ solution (73.5 to 147 μl) and the sterile concentrated carrageenan base (0.5 g) are mixed with syringes as described in Example 1. The pH is adjusted to 7.0 with the addition of 60 μL HCl 1 N. This fibronectin-PDGF-carrageenan dressing is then placed in a borosilicate glass vial (5 mL for a surface area of 5.3 cm²) and frozen at −20° C. for 2 hours and 30 minutes at −80° C. The water is then removed by freeze-drying using a Labconco freeze-dryer (model 77580, Kansas City, Mo.). By this technique, a 4.2 cm² fibronectin-PDGF-ββ-carrageenan wound dressing with a sponge-like structure is produced, with a concentration of up to 80 μg/mm² fibronectin and with a rhPDGF-ββ concentration ranging from 0.17 to 0.35 μg/mm².

### EXAMPLE 10

### Fibronectin/rhKGF-2 Calcium-Alginate Dressing Composition

In another embodiment of this invention, the DERMALINK delivery system containing various amounts of Keratinocyte Growth Factor-2 (KGF-2) is prepared as follows: 1 g of sodium alginate (Pronova UP LVG, Pronova Biomedical A.S., Oslo, Norway) is dispersed in 99 g of tap water demineralized using Millipore Milli-Q water system (MQW) containing 150 μL of glacial acetic acid with a paddle type stirrer for about 1 hour at 50° C. This dispersion provides a 100 mL of alginate solution 1% (w/w) at pH 4.2 which is then sterilized by filtration under 0.22 μm PVDF filter (Millex-GV, Millipore Corp., Bedford, Mass.).

The pH of 10 mL MQW is adjusted at pH 11.6 with the addition of 23 μL of NaOH 3M. The lyophilized fibronectin (0.1 g) is next dissolved in this MQW pH 11.6 under gentle agitation. The solution is maintained at 37° C. until complete solubilization of fibronectin occurs. This solution is then filtered through a 0.22 μm PVDF filter (Millex-GV, Millipore Corp., Bedford, Mass.).

In a preferred embodiment, lyophilized KGF-2 (Repifermin, Human Genome Sciences, Inc. Rockville, Md.) is first dissolved in MQW at a fixed concentration of 1 mg/mL and then filter sterilized as for the fibronectin solution. Other sources and forms of KGF-2 can also be used. The 10 μg/ml sterile solution of fibronectin (3.4 ml)), the 1 mg/ml KGF-2 solution (42 to 252 μl) and the sterile-filtered 1% solution sodium alginate (1.6 mL; pH 4.2) are then mixed into syringes as described in Example 1. Gellation of the solution is achieved by the addition of 15 μL 0.2 M NaCl+0.2 M CaCl₂. At this point, the fibronectin-KGF-2-calcium alginate complex forms an emulsion-like solution at a pH between 5.0 and 5.2. This solution is then placed in a borosilicate glass vial (5 mL for a surface area of 5.3 cm²) and frozen at −20° C. for 2 hours and 30 minutes at −80° C. The water is then removed by freeze-drying using a Labconco freeze-dryer (model 77580, Kansas City, Mo.). By this technique, a 4.2 cm² solid sponge-like fibronectin-KGF-2-calcium alginate delivery system (DERMALINK™ delivery system containing KGF-2) with a fibronectin concentration up to 80 μg/mm and with a KGF-2 concentration ranging from 0.1 to 0.6 μg/mm is produced.

### EXAMPLE 11

### Fibronectin/rhPDGF-ββ/rhKGF-2 Calcium-Alginate Dressing Composition

The DERMALINK delivery system may also be prepared containing various amounts of rhPDGF-ββ and various amounts rhKGF-2 as follows: 1 g of sodium alginate (Pronova UP LVG, Pronova Biomedical A.S., Oslo, Norway) is dispersed in 99 g of tap water demineralized using Millipore Milli-Q water system (MQW) containing 150 μL of glacial acetic acid with a paddle type stirrer for about 1 hour at 50° C. This dispersion provides a 100 mL of alginate solution 1% (w/w) at pH 4.2 which is then sterilized by filtration under 0.22 μm PVDF filter (Millex-GV, Millipore Corp., Bedford, Mass.).

The pH of 10 mL MQW is adjusted at pH 11.6 with the addition of 23 μL of NaOH 3M. The lyophilized fibronectin (0.1 g) is next dissolved in this MQW pH 11.6 under gentle agitation. The solution is maintained at 37° C. until complete solubilization of fibronectin occurs. This solution is then filtered through a 0.22 μm PVDF filter (Millex-GV, Millipore Corp., Bedford, Mass.).

Lyophilized rhPDGF-ββ and rhKGF-2, from sources as described in the previous examples are each dissolved in MQW to a final concentration of 1 mg/ml then filter sterilized as for the fibronectin solution. The 10 mg/ml sterile solution of fibronectin (3.4 ml)), the 1 mg/ml rhPDGF-ββ solution (73.5 to 147 μl), the 1 mg/ml rhKGF-2 solution (42 to 252 μl) and the sterile-filtered 1% solution sodium alginate (1.6 mL; pH 4.2) are then mixed into syringes as described in Example 1. Gellation of the solution is achieved by the addition of 15 μL 0.2 M NaCl+0.2 M CaCl₂. At this point, the fibronectin-rhPDGF-ββ-rhKGF-2-calcium alginate complex forms an emulsion-like solution at a pH between 5.0 and 5.2. This solution is then placed in a borosilicate glass vial (5 mL for a surface area of 5.3 cm²) and frozen at −20° C. for 2 hours and 30 minutes at 80° C. The water is then removed by freeze-drying using a Labconco freeze-dryer (model 77580, Kansas City, Mo.). By this technique, a 4.2 cm² solid sponge-like fibronectin-rhPDGF-ββ-rhKGF-2-calcium alginate delivery system (DERMA-LINK™ delivery system containing rhPDGF-ββ and rhKGF-2) is produced, containing a fibronectin concentration of up to 80 μg/mm², a rhPDGF-ββ concentration ranging from 0.17 to 0.35 μg/mm² and a rhKGF-2 concentration ranging from 0.1 to 0.6 μg/mm².

### EXAMPLE 12

### Wound Healing Promoters Other Than or in Addition to Fibronectin

The solid wound formulations of the invention can include other wound healing promoters having a composition similar to fibronectin, such as proteins of similar size (extracellular matrix proteins, e.g. thrombospondin, laminin, vitronectin, fibrinogen) or similar to PDGF-ββ and KGF-2 (including peptides and growth factors such as KGF (FGF-7, acidic FGF, basic FGF, TGF-β, IGF-I, IGF-II, VEGF and GM-CSF or their analogs). In preferred embodiments of the invention, the appropriate species-specific wound healing promoters are used, i.e., human fibronectin and/or other wound healing promoters for human applications, equine fibronectin and/or other wound healing promoters for equine applications.

Although the present invention has been described in relation to particular embodiments thereof, many other variations, modifications, and uses will become apparent to those skilled in the art. It is therefore understood that numerous variations of the invention can be made which are well within the scope and spirit of this invention as described in the appended claims.

Cited References

| U.S. Pat. Nos. | | Published | Inventor(s) |
|---|---|---|---|
| 721355 | EP | February 1998 | Kershaw |
| 0113967 | WO | March 2001 | Beaulieu et al. |
| 9008833 | WO | August 1990 | Guan and Hynes |
| 5877149 | US | March 1999 | Beaulieu |
| 4766073 | US | August 1998 | Murray et al. |
| 5428135 | US | June 1995 | Lyons et al. |
| 5149792 | US | September 1992 | Thomason |
| 5453489 | US | September 1995 | Ruoslathi |
| 5958874 | US | September 1999 | Clark |
| 5922676 | US | July 1999 | Pasqualini |
| 5629287 | US | May 1997 | Brown and Blun |
| 5610148 | US | March 1997 | Brown |
| 4973466 | US | November 1990 | Reich |
| 5516896 | US | May 1996 | Murray et al. |
| 5905142 | US | May 1999 | Murray |
| 5187263 | US | February 1993 | Murray et al. |
| 5759815 | US | June 1998 | Charette et al. |

Other References

Alexander, D. M. et al., (1992) Prot. Expres. Purif. 3, 204-211

Bennett, N. T. and Schultz, G. S. (1993a) Am. J. Surg. 165, 728-737

Bennett, N. T. and Schultz, G. S. (1993a) Am. J. Surg. 165,74-81

Brotchie, H. and Wakefield, D. (1990) Australas. J. Dermatol. 31, 47-56

Cooper D. M. et al. (1994) Ann. Surg. 219, 688-691

Deuel, T. F. et al. (1981) J. Biol. Chem. 256, 8896-8899.

Greenhalgh, D. G. et al. (1990) Am. J. Pathol. 136, 1235-1246

Grinnell, F. et al. (1992) J. Invest. Dermatol. 98, 410-416

Heldin, C. H. et al. (1979) Proc. Natl Acad. Sci. U S A 76, 3722-3726

Hoppe, J. et al. (1989) Biochemistry 28, 2956-2960

Horowitz, B. and Chang, M. D. Y. (1989) In: Fibronectin, Mosher, D. F., ed., San Diego, Academic Press

Horowitz, B. et al. (1992), Blood 79, 826-831

Hynes, R. O. (1990) Fibronectins. Rich, A., ed., New-York, Springer-Verlag

Jimenez, P. A. and Rampy, M. A. (1999) J. Surg. Res. 81, 238-242

Johnsson, A. et al. (1984) EMBO J. 3, 921-928

Kornblihtt, A. R. et al. (1985) EMBO J. 4, 1755-1759

Mustoe, T. A. et al. (1991) J. Clin. Invest. 87, 694-703

Nishida, T., et al. (1984) Arch. Ophtalmol. 102, 455-456

Phan, T. M. et al., (1987) Am. J. Ophthalmol. 104, 494-501

Pierce, G. F. et al. (1988) J. Exp. Med. 167, 974-987

Potts, J. R. and Campbell, I. D. (1994) Curr. Opin. Cell Biol. 6, 648-655

Robson, M. C. (1996) Prog. Dermatol. 30, 1-7

Robson, M. C. et al.(1998) Am. J. Surg. 176 (suppl. 2A): 80S-82S

Singer, A. J. and Clark, R. A. F. (1999) New Engl. J. Med. 341, 738-745

Wysocki, A. et al., (1988) Arch. Dermatology 124, 175-177

Xia, Y. P. et al. (1999) J. Pathol. 188, 431-438.

We claim:

1. A solid wound formulation comprising:
at least 34-65% fibronectin by weight of the solid wound formulation and
at least one growth factor, wherein the growth factor is selected from the group consisting of platelet-derived growth factor (PDGF), keratinocyte growth factor (KGF), keratinocyte growth factor-2 (KGF-2), granulocyte-macrophage colony stimulating factor (GM-CSF), vascular endothelial growth factor (VEGF), insulin-like growth factor (IGF), transforming growth factors (TGF), epidermal growth factor (EGF) and fibroblast growth factors (FGF).

2. The solid wound formulation according to claim 1 wherein the fibronectin comprises plasma fibronectin, recombinant fibronectin, biologically active fragments of plasma fibronectin, and biologically active fragments of recombinant fibronectin.

3. The solid wound formulation according to claim 1 wherein the fibronectin is human fibronectin.

4. The solid wound formulation according to claim 1 wherein the fibronectin is fibronectin from a non-human animal.

5. A solid wound formulation comprising:
at least 34-65% fibronectin by weight of the solid wound formulation and
the growth factor is PDGF.

6. The solid wound formulation of claim 5 wherein the PDGF is human PDGF.

7. The solid wound formulation of claim 5 wherein the PDGF is non-human PDGF.

8. A solid wound formulation comprising:
at least 34-65% fibronectin by weight of the solid wound formulation and
PDGF polypeptides wherein the PDGF polypeptides are selected from the group consisting of full-length PDGF, PDGF-ββ, PDGF-αβ, PDGF-αα, recombinant PDGF-ββ, recombinant PDGF-αβ, recombinant PDGF-αα or biologically active fragments thereof.

9. The solid wound formulation of claim 8 wherein the PDGF polypeptides are human PDGF polypeptides.

10. The solid wound formulation of claim 8 wherein the PDGF polypeptides are non-human PDGF polypeptides.

11. The solid wound formulation of claim 5 which contains up to about 0.27 to about 0.32% PDGF by weight of the solid wound formulation.

12. A solid wound formulation comprising:
at least 34-65% fibronectin by weight of the solid wound formulation and
the growth factor is KGF-2.

13. The solid wound formulation of claim 12 wherein the KGF-2 is human KGF-2.

14. The solid wound formulation of claim 12 wherein the KGF-2 is non-human KGF-2.

15. The solid wound formulation of claim 1 wherein the fibronectin and the growth factor retain structural and functional integrity after the fibronectin and the growth factor are incorporated into the formulation.

16. A pharmaceutical delivery system comprising a fibrous dressing, which releases:
i) an effective amount of fibronectin and
ii) an effective amount of a growth factor,
wherein the fibronectin and the growth factor are released from the dressing into the wound when the dressing is applied to the wound.

17. The pharmaceutical delivery system according to claim 16 wherein the fibrous dressing comprises a plant polysaccharide.

18. The pharmaceutical delivery system according to claim 17, wherein the plant polysaccharide is selected from the group consisting of alginates, carrageenans, cellulose derivatives and oxidized cellulose derivatives.

19. The pharmaceutical delivery system according to claim 16, wherein the fibrous dressing comprises a tissue matrix system.

20. The pharmaceutical delivery system according to claim 16, wherein the fibrous dressing is a solid before contact with an exudating wound and is at least partially a gel after contact with an exudating wound.

21. A pharmaceutical delivery system comprising a fibrous dressing, which releases:
i) 30 to 160 μg/mm² of fibronectin and
ii) 0.1 to 1.0 μg/mm² of PDGF-ββ,
wherein the fibronectin and the PDGF-ββ are released from the dressing into the wound when the dressing is applied to the wound.

22. A pharmaceutical delivery system comprising a fibrous dressing, which releases:
i) 35 to 90 μg/mm² of fibronectin and
ii) 0.15 to 0.6 μg/mm² of PDGF-ββ,
wherein the fibronectin and the PDGF-ββ are released from the dressing into the wound when when the dressing is applied to the wound.

23. A pharmaceutical delivery system comprising a fibrous dressing, which releases:
i) about 80 μg/mm² of fibronectin and
ii) about 0.35 μg/mm² of PDGF-ββ,
wherein the fibronectin and the PDGF-ββ are released from the dressing into the wound when the dressing is applied to the wound.

24. A pharmaceutical delivery system comprising a fibrous dressing, which releases:
i) 30 to 160 μg/mm² of fibronectin and
ii) 0.1 to 0.6 μg/mm²of KGF-2,
wherein the fibronectin and the KGF-2 are released from the dressing into the wound when the dressing is applied to the wound.

25. A pharmaceutical delivery system comprising a fibrous dressing, which releases:
i) 30 to 160 μg/mm² of fibronectin,
ii) 0.1 to 1.0 μg/mm² of PDGF-ββ,
iii) 0.1 to 0.6 μg/mm² of KGF-2,
wherein the fibronectin, the PDGF-ββ and the KGF-2 are released from the dressing into the wound when the dressing is applied to the wound.

26. The pharmaceutical delivery system according to claim 17, comprising a fibronectin-polysaccharide dressing wherein at least 80% of the fibronectin is absorbed in a dermal layer of a deepithelialized skin diffusion cell system after 12 hours.

27. The pharmaceutical delivery system according to claim 16, further comprising an effective amount of a wound healing promoter other than fibronectin.

28. The pharmaceutical delivery system according to claim 27, wherein the wound healing promoter other than fibronectin is selected from the group consisting of thrombospondin, laminin, vitronectin, fibrinogen or fibrin.

29. The pharmaceutical delivery system according to claim 16, wherein the growth factor is selected from the group consisting of platelet-derived growth factor (PDGF), keratinocyte growth factor (KGF), keratinocyte growth factor-2 (KGF-2), granulocyte-macrophage colony stimulating factor (GM-CSF), vascular endothelial growth factor (VEGF), insulin-like growth factor (IGF), transforming growth factors (TGF), epidermal growth factor (EGF) and fibroblast growth factors (FGF).

30. The pharmaceutical delivery system according to claim 16 wherein the growth factor comprises a full length PDGF polypeptide.

31. The pharmaceutical delivery system according to claim 30, wherein the PDGF is selected from the group consisting of PDGF-ββ, PDGF-αβ, PDGF-αα, recombinant PDGF-ββ, recombinant PDGF-αβ, recombinant PDGF-αα or biologically active fragments thereof.

32. The pharmaceutical delivery system according to claim 31 wherein the PDGF is human PDGF.

33. The pharmaceutical delivery system according to claim 31 wherein the PDGF is non-human PDGF.

34. A method of producing a solid wound dressing according to claim 1 comprising the steps of
i) mixing a dispersion of insoluble fibers, a solution of fibronectin and a solution of a growth factor to produce a homogeneous mixture; and
ii) freeze-drying the homogeneous mixture of step i) to produce a solid wound dressing.

35. The method according to claim 34, wherein the dispersion of insoluble fibers contain some soluble fibers.

36. The method according to claim 35, wherein the insoluble fibers can soluble under some ionic conditions.

37. The method according to claim 34, wherein the solution of fibronectin has a concentration of 10 mg/mL.

38. The method according to claim 34, wherein steps i) and ii) are conducted under sterile conditions and comprising the further step of:
iii) placing and sealing the solid wound dressing of step ii) in a sterile container under sterile conditions.
